(19) United States
(12) Patent Application Publication
(10) Pub. No.: US 2026/0272349 A1
(43) Pub. Date: Sep. 17, 2026

(54) CANNULA SET LOADING UNIT, CANNULA SET LOADING DEVICE AND CANNULA INSERTION SYSTEM

(71) Applicant: Vitestro Holding B.V., Utrecht (NL)

(72) Inventors: Toon Olaf Overbeeke, Utrecht (NL); Brian Robert Joseph, 'S-Gravenhage (NL); Johannes Gerhard Daniël Karssen, Utrecht (NL); Arris Cornelis Jonker, Utrecht (NL); Hiddo Johan During, Utrecht (NL); Marc Boele Jan de Lange, Utrecht (NL)

(21) Appl. No.: 18/868,615

(22) PCT Filed: May 26, 2023
(86) PCT No.: PCT/EP2023/064230

§ 371 Date: Nov. 22, 2024

(30) Foreign Application Priority Data
May 27, 2022 (NL) 2032010

(51) Int. Cl. A61B 5/15; A61B 5/154; A61B 50/20

(52) U.S. Cl. CPC A61B 5/150748; A61B 5/15003; A61B 5/150343; A61B 5/150473; A61B 5/150572; A61B 5/150732; A61B 5/150893; A61B 5/1509; A61B 5/154; A61B 50/20

(57) ABSTRACT

The present invention comprises a cannula set loading unit to load a cannula set into a cannula insertion system, comprising: a cannula set having at least one patient needle for introduction into a patient, a cartridge having a predefined location for holding the patient needle in a fixed position with respect to the cartridge. The invention further provides a cannula set loading device to load such cannula set loading unit into a cannula insertion system.

### CROSS-REFERENCE TO RELATED APPLICATION(S)

The present application claims the benefit of priority to Dutch Patent Application No. 2032010, filed May 27, 2022, which is incorporated by reference herein in its entirety.

### TECHNICAL FIELD

The present invention relates to a cannula set loading unit to load a cannula set into a cannula insertion system. The invention further relates to a cannula set loading device to load a cannula set in a cannula insertion system and a cannula insertion system to insert a cannula into a human or animal body.

### BACKGROUND

There are various scenarios in which it may be useful to administer fluids to a patient and/or withdraw fluids from a patient using a cannula set. For example, a cannula set for venipuncture can be used to withdraw blood from a patient (e.g., for blood testing purposes) via a patient needle that is introduced into a vein of a patient.

For venipuncture, cannula sets can be manipulated manually, such as handled manually by a technician or clinician who selects a cannula set, manipulates the patient needle of the cannula set into position, inserts the patient needle into the patient and coordinates a blood draw through the patient needle to one or more collection tubes, and removes the patient needle from the patient. While some automatic venipuncture systems exist, there still limitations around properly loading cannula set(s) for automated manipulation, among other challenges. For example, operator involvement may still be required to select and/or load a cannula set for use by an automatic venipuncture system.

Thus, there is a need for new and improved system(s) for selecting and/or loading a cannula set for use with a patient, such as by an automated venipuncture system.

### SUMMARY

In cannula insertion systems, for example automatic venipuncture systems, cannula sets are used. Such cannula sets typically comprise a patient needle to be introduced into a patient. The cannula set is normally disposable and is discarded after use.

In a cannula insertion system the cannula set, in particular the patient needle, may be connected to a cannula insertion device to enable the cannula insertion device to introduce the patient needle into a patient. After use, the cannula may be released from the cannula insertion device and discarded. In particular in an automatic cannula insertion system, it is desirable that the cannula set may be automatically loaded into the cannula insertion system.

CN106580344A discloses an automatic venipuncture robot with a cannula set supply machine to automatically provide cannula sets. This cannula set supply machine comprises a strip on a reel. On the strip a plurality of cannula sets is mounted. The cannula sets may be moved by movement of the strip to a release position where a respective cannula set may be taken from the strip and introduced into the cannula insertion system.

A drawback of this cannula set supply machine is that the position of any particular cannula set is not properly defined. It may be difficult to find the actual position of the cannula set to enable the cannula insertion system to connect the patient needle to the cannula insertion device. Moreover, the cannula set supply machine of CN106580344A is a bulky device. It may be difficult to integrate this cannula set supply machine in an actual automatic cannula insertion system.

It is an object of the invention to provide a cannula insertion system that provides an improved supply of cannula sets for a cannula insertion system. In particular, it is an object of the invention to provide a supply of cannula sets for a cannula insertion system that enables a reliable introduction of the cannula set into the cannula insertion system and/or that provides a relatively compact system to supply cannula sets into the cannula insertion system.

The invention provides a cannula set loading unit to load a cannula set into a cannula insertion system, the cannula set loading unit comprising:
a cannula set having at least one patient needle for introduction into a patient.
a cartridge having a predefined location for holding the patient needle in a fixed position with respect to the cartridge.

The cartridge provides a predefined location for holding at least a portion of a cannula set, such as the patient needle. For example, the cartridge can be configured to hold the patient needle in a predefined, fixed position with respect to the cartridge, through one or more cartridge features. At the same time, the position of the cartridge can be precisely controlled or determined by a cannula loading device, e.g., by mechanical alignment and/or by one or more sensors measuring the position of the cartridge. As a result, the position of the cannula set with respect to the cannula set loading device may be well-defined. This allows the cannula set loading device to effectively take the cannula set and load it into the cannula insertion system in a controlled manner.

Further, a cartridge can be easily manipulated, packaged, and transported with a low risk that the cannula set will be released from or displaced on the cartridge. The cartridge also enables efficient placement of one or more cannula set loading units in a storage of a cannula set loading device.

The cartridge may have one or more fixtures (e.g., protrusions and/or openings) to define locations for holding at least a portion of a cannula set, such as the patient needle. The cartridge may also have protrusions and/or openings to align the cartridge with respect to another object, for example a cannula set loading device or another cartridge.

In some embodiments, the cartridge may include and/or may be made of rigid material, for example a plastic material or metal. The cartridge may be injection-moulded, machined, 3D-printed, and/or made with any suitable manufacturing techniques. In some embodiments, the cartridge may include another material sufficiently rigid for maintaining a sufficiently fixed positional relationship between the patient needle of the cannula set and the cartridge. For example, the cartridge may include a malleable material (e.g., paper, cardboard, cardstock, flexible plastic, etc.) in which one or more fixtures for holding at least a portion of the cannula set are formed by folding, cutting, and/or the like.

The cartridge may support one cannula set or multiple cannula sets.

The cartridge may have any suitable size and shape for supporting one or more cannula sets. For example, in some embodiments, the cartridge may have a generally rectangular shape, a square shape, a circular shape, and/or or the like. In some embodiments, the cartridge may, for example, include a tray with one or more fixtures for holding at least a portion of a cannula set, such as the patient needle. In some embodiments, the cartridge may include a bracket such as a frame with one or more fixtures for holding at least a portion of a cannula set.

The cartridge may be disposable or may be reusable, for example after sterilisation of the cartridge. An operator can load each cartridge with a respective cannula set to form a cannula set loading unit that is stocked and ready for use with the cannula insertion system. A cartridge-loading process can be performed for cartridges that are disposable (e.g., single one-time insertion of a cannula set onto a single-use, disposable cartridge), or reusable (e.g., multiple, repeated insertion of a cannula set onto a reusable cartridge). In some embodiments, during a cartridge-loading process, a cartridge can be outside of (e.g., removed from, separated from) the cannula insertion system in which the cartridge is to be used. Additionally or alternatively, during a cartridge-loading process, a cartridge can be retained in or with the cannula insertion system. For example, one or more cartridges can be fixedly coupled to, removably coupled to, or integrated within the cannula insertion system, and accessed by an operator for loading with a cannula set. As an illustrative example embodiment, the one or more cartridges can be accessed through a door or other opening, or can be accessed in a portion of the cannula insertion system that is extendable outside a housing of the cannula insertion system.

In an embodiment, the cartridge is designed to hold the patient needle in a predefined position and orientation. A predefined position and orientation of the patient needle with respect to the cartridge may be advantageous, for example to connect the patient needle to a cannula insertion device. Due to the predefined position and orientation of the patient needle the position of the bevel on the needle end of the patient needle is also known. For example, it may be advantageous for the cartridge to hold the patient needle with its bevel in a predefined orientation and/or position, such that a cannula set loading device can load the patient needle into the cannula insertion system with the bevel facing upwards, which can be consistent with a desired orientation of the patient needle during patient needle insertion.

In an embodiment, the patient needle comprises a patient needle hub to connect the patient needle to a cannula insertion device of the cannula insertion system. The patient needle hub can be used to releasably connect the patient needle to the cannula insertion device. The patient needle hub may also be used to manipulate the patient needle, for example to take the patient needle from the cartridge.

In an embodiment, the cartridge is stackable with another cartridge. For example, the cartridge can be vertically stackable on another cartridge, horizontally stackable adjacent to another cartridge, or stacked at an angle adjacent to another cartridge. By stacking the cartridges, a stack of cannula set loading units may be provided. Such stacking facilitates efficient handling of the cannula set loading units, for example when loading a number of cannula set loading units in a storage of a cannula set loading device.

In some embodiments, the cartridge comprises one or more snap connection elements configured to connect the cartridge to the other cartridge (e.g., snap the cartridge to the other cartridge). The one or more snap connection elements are for example snap protrusions on one side of the cartridge and snap recesses on the side of the other cartridge facing the one side of the cartridge. By creating snap connections between different cartridges of a stack of cartridges, a stack of cartridges can be handled as a single package, for example during loading into a storage of a cannula set loading device. This may make loading of cartridges into the storage more efficient.

In an embodiment, the cannula set comprises a first needle cap, the first needle cap being configured to receive at least a portion (e.g., a distal needle end) of the patient needle, such that at least a portion of the patient needle is covered or enclosed by the first needle cap. By mounting a first needle cap on at least a needle end of the patient needle, the patient needle can be kept in a sterile, closed environment. Before the patient needle can be used, the needle end of the patient needle should be taken out of the first needle cap (or the first needle cap should be otherwise removed or decupled from the patient needle). Additionally or alternatively, the cartridge can include the first needle cap for receiving at least a portion of the patient needle, such that at least a portion of the patient needle is covered or enclosed by the first needle cap. For example, the first needle cap can be integrated with the body of the cartridge (e.g., the cartridge can include a recess configured to receive at least a portion of the patient needle). In some embodiments, a first portion of the first needle cap can be part of the cannula set, and a second portion of the first needle cap can be part of the cartridge, such that the first and second portions of the first needle cap can collectively cover or enclose at least a portion of the patient needle. The first and second portions of the first needle cap can, for example, couple together (e.g., via a snap fit connection or other mating features), and/or overlap (e.g., via overlapping coaxial sleeves) to collectively cover at least a sufficient portion of the patient needle.

The first needle cap (whether part of the cannula set and/or the cartridge) and the patient needle may be configured to hold the patient needle in a fixed position and orientation with respect to the first needle cap.

In an embodiment, the first needle cap may be connected to or integrated in the cartridge such that the first needle cap will remain on the cartridge when the patient needle is taken out of the first needle cap. In another embodiment, the first needle cap may be removably coupled to the cartridge, such that the first needle cap can be taken by a cannula set loading device system from the cartridge together with the rest of the cannula set and later removed from the patient needle.

In an embodiment, the first needle cap is releasably coupled to the cartridge, for example held by a snap connection on the cartridge. The advantage of the snap connection is that the first needle cap can be mounted over the needle end before the cannula set is mounted on the cartridge. The snap connection can be used to connect the first needle cap on a fixed position on the cartridge. Advantageously, the snap connection is configured to fix the first needle cap in six degrees of freedom with respect to the cartridge, and the patient needle and the first needle cap are configured to hold the patient needle in a fixed position and orientation with respect to the first needle cap. As a result, the position of the patient needle with respect to the cartridge is defined when the first needle cap is mounted on the snap connection of the cartridge. This has the advantage that the position of relevant parts of the patient needle such as the patient needle hub and bevel of the needle end of the patient needle are known.

The snap connection may for example have a number of extensions that can hold the first needle cap. The first needle cap and the extensions may have a contact surfaces that ensure that the first needle cap can only be mounted in one fixed position with respect to the cartridge.

In an embodiment, a tamper indicator, such as a tamper evident seal or other tamper evident, is provided between the patient needle and the first needle cap. Such a tamper indicator may indicate whether the patient needle has been taken out of the first needle cap (e.g., whether the patient needle has been decoupled from the first needle cap). If the tamper indicator is not in its original state this may indicate that there is no guarantee that needle is sterile, and such a needle should not be used for insertion into the human or animal body. In some embodiments, the tamper indicator can include one or more breakable (or otherwise disruptable) elements attached to the patient needle and the first needle cap. When the patient needle and the first needle cap are separated, one or more of the following types of breaking may occur: (i) the breakable element may become deformed (e.g., stretched, partially or fully torn, or otherwise separated); (ii) the breakable element may partially or fully detach from the patient needle; and/or (iii) the breakable element may partially or fully detach from the first needle cap. Such breaking of an element can indicate a possibly non-sterile patient needle. The tamper indicator may for example include a strip or ring of paper or other suitable material connected to the patient needle and the first needle cap. When the patient needle is pulled out of the first needle cap, the strip or ring may be torn and therefore not hamper the ease of use of the patient needle and the first needle cap. Additionally or alternatively, the tamper indicator may include a partially or fully circumferential seal connecting the patient needle and the first needle cap. For example, the seal connecting the patient needle and first needle cap may include a plastic weld connecting the first needle cap and a hub of the patient needle. When the patient needle is pulled out of the first needle cap, the seal may be broken and allow the patient needle and the first needle cap to separate.

In some embodiments, the cannula set may comprise a tube connected to the patient needle. In such embodiments, the cartridge may also have a predefined location for holding the tube in a fixed position with respect to the cartridge. For example, the cartridge may have one or more features (e.g., protrusions, openings, etc.) for holding the tube in a predefined, fixed arrangement within or on the cartridge.

In some embodiments, the cannula set comprises a connection element configured to connect the cannula set to a further device. In some embodiments, the connection element can include a fluid tight connector (e.g., Luer lock) or a needle such as a collection tube needle configured to connect the cannula set to a fluid collection tube (e.g., blood collection tube). The patient needle and the connection element may be coupled to a rigid body and extend in different directions from each other. For example, the cannula set can include a rigid body with a patient needle (which may be configured for insertion in a patient) extending from the rigid body in one direction, and a tube needle such as a collection tube needle (which may be configured for insertion in a blood collection tube) extending from the rigid body in a different direction than the patient needle. For example, a distal end of the patient needle and a distal end of the blood patient needle can be oriented in opposite directions. In some of these embodiments, the cannula set can lack a separate tube connecting the patient needle and the connection element. For example, in some embodiments, the patient needle and the connection element (e.g., tube needle) can have generally fixed positions and/or orientations relative to each other.

In some embodiments, the cannula set comprises a tube and a connection element, wherein the patient needle is connected at one end of the tube and the connection element is connected at an opposite end of the tube to connect the tube to a further device. The tube may, for example, be flexible to allow for different relative positions of the patient needle and the connection element. When the patient needle is brought into a human or animal body, the tube may be used to guide fluid into or from the human or animal body. This fluid may be guided via the connection element from or towards the further device. For example, when the cannula set is used for infusion, the connection element of the cannula set may be connected to an infusion device to bring a fluid into the human or animal body. The connection element can be any element to bring the tube into fluid connection with another device. The connection element may for example a fluid tight connector, such as a Luer lock, or a needle that can be moved through a sealing element of a further device.

In an embodiment, the connection element is a collection tube needle arranged to connect the cannula set to a blood collection tube. The cannula set may be used in a cannula insertion system configured for automatic blood withdrawal of a patient. To guide the blood into a blood collection tube, the connection element may be a tube needle to provide a fluid connection to a blood collection tube. Any other connection element to the blood collection tube may also be used.

In an embodiment, the cartridge is designed to hold the tube needle in a predefined position and orientation. A predefined position and orientation of the tube needle with respect to the cartridge is advantageous, for example to connect a tube needle hub of the tube needle to a tube needle hub manipulator.

In an embodiment, the cannula set comprises a second needle cap configured to receive at least at least a portion (e.g., a distal needle end) of the tube needle, such that at least a portion of the tube needle is covered or enclosed by the second needle cap. Similar to the patient needle, the cannula set may comprise a second needle cap to keep the needle end of the tube needle in a closed, sterile environment until use. The first and/or second needle caps may be arranged on the patient needle and tube needle, respectively, before the cannula set is mounted on the cartridge.

Additionally or alternatively, the cartridge can include the second needle cap for receiving at least a portion of the tube needle, such that at least a portion of the tube needle is covered or enclosed by the second needle cap. For example, the second needle cap can be integrated with the body of the cartridge (e.g., the cartridge can include a recess configured to receive at least a portion of the tube needle). Furthermore, in some embodiments, a first portion of the second needle cap can be part of the cannula set, and a second portion of the second needle cap can be part of the cartridge, such that the first and second portions of the second needle cap can collectively cover or enclose at least a portion of the patient needle. The first and second portions of the second needle cap can, for example, couple together (e.g., via a snap fit connection or other mating features), and/or overlap (e.g., via overlapping coaxial sleeves) to collectively cover at least a sufficient portion of the tube needle.

In some embodiments, the second needle cap may be connected to or integrated in the cartridge such that the second needle cap will remain on the cartridge when the tube needle is taken out of the second needle cap. Alternatively, in some embodiments, the second needle cap may be removably coupled to the cartridge, such that the second needle cap can be taken by a cannula set loading device system from the cartridge together with the rest of the cannula set and later removed from the tube needle.

In some embodiments, by mounting the first needle cap on at least the needle end of the patient needle and the second needle cap on at least the needle end of the tube needle, the whole fluidic path between the patient needle and the tube needle can be kept in a closed, sterile environment.

In an embodiment, the first needle cap may close the patient needle and the connection element may be closed by a connection element closure element, such that the whole fluidic path from the patient needle to the connection element will be kept in a closed, sterile environment. This may obviate the need of a separate sterile pouch in which the cannula set is placed.

In an embodiment, the second needle cap is releasably coupled to the cartridge, for example held by a snap connection on the cartridge. Similar to the patient needle, the tube needle may be arranged in a fixed position and orientation with respect to the second needle cap and the second needle cap may be mounted in a fixed position in six degrees of freedom on the cartridge, such that the tube needle is arranged in a fixed position and orientation with respect to the cartridge.

In an embodiment, a tamper indicator, such as a tamper evident seal or other tamper evident, is provided between the tube needle (or other connection element) and the second needle cap. Similar to the tamper indicator described above with respect to the patient needle and the first needle cap, the tamper indicator for the tube needle and the second needle cap may indicate whether the tube needle has been taken out of the second needle cap (e.g., whether the tube needle has been decoupled from the second needle cap). If the tamper indicator is not in its original state this may indicate that there is no guarantee that needle is sterile, and such a needle should not be used for connecting the cannula set to a collection tube or other further device. In some embodiments, the tamper indicator can include one or more breakable (or otherwise disruptable) elements attached to the tube needle and the second needle cap. Similar to that described above with respect to the tamper indicator for the patient needle and the first needle cape, when the tube needle and the second needle cap are separated, one or more of the following types of breaking may occur: (i) the breakable element may become deformed (e.g., stretched, partially or fully torn, or otherwise separated); (ii) the breakable element may partially or fully detach from the tube needle; and/or (iii) the breakable element may partially or fully detach from the second needle cap. Such breaking of an element can indicate a possibly non-sterile tube needle. The tamper indicator may for example include a strip or ring of paper or other suitable material connected to the tube needle and the second needle cap. When the tube needle is pulled out of the second needle cap, the strip or ring may be torn and therefore not hamper the ease of use of the tube needle and the second needle cap. Additionally or alternatively, the tamper indicator may include a partially or fully circumferential seal connecting the tube needle and the second needle cap. For example, the seal connecting the tube needle and second needle cap may include a plastic weld connecting the second needle cap and a hub of the tube needle. When the tube needle is pulled out of the second needle cap, the seal may be broken and allow the tube needle and the second needle cap to separate.

In an embodiment, the connection element comprises a connection element hub to manipulate the connection element. To couple the connection element to a further device, the connection element may comprise a connection element hub that allows manipulation of the connection element. The connection element hub may for example be a cylindrical part between two flanges arranged to accommodate a connection element manipulator, for example a fork with two prongs, wherein the prongs can be arranged at opposite sides of the cylindrical part between the two flanges.

As described above, in some embodiments, the cannula set comprises a tube connected to the patient needle and/or a connection element. In such embodiments, the cartridge may be configured to hold the tube in a predefined, fixed arrangement (e.g., position, orientation, shape, etc.) with respect to the cartridge. For example, the cartridge may be designed to receive and hold the tube in a loop. When the cannula set is mounted on the cartridge, a tube of the cannula set may be arranged in a loop defined by the cartridge such that the loop runs along a fixed path of the cartridge.

The invention further provides a cannula set loading device to load a cannula set into a cannula insertion system. The cannula set loading device may be configured to take a cannula set from a cannula set loading unit and to connect at least the patient needle of the cannula set to the cannula insertion system.

In an embodiment, the cannula set loading device comprises a storage for supporting a stack of cannula set loading units. The storage can be used to store multiple cannula set loading units, such that multiple cannula sets are ready for use and can be automatically loaded into the cannula insertion system. The stack of cannula set loading units can, for example, be generally arranged vertically, horizontally, or at any suitable angle. Additionally or alternatively, the cannula set loading units can be arranged relative to each other in any suitable manner in the stack (e.g., fully face-to-face, partially face-to-face such as in a staggered manner, etc.) New cannula set loading units may be placed in the storage when needed. The storage may comprise an opening which is accessible from the exterior of the cannula set loading device in order to place new cannula set loading units into the cannula set loading device.

In an embodiment, for example if the cannula set loading device does not comprise a storage or if it is desirable that cannula set loading unit may also be directly loaded into the cannula set loading device, the cannula set loading device may comprises a cannula set loading port, through which for each procedure a new cannula set loading unit may be introduced into the cannula set loading device.

In an embodiment, the cannula set loading device comprises a transfer device to transfer a cannula set loading unit from a storage or introduction position to a loading position. The transfer device is arranged to move the cannula set loading unit from a storage or introduction, in which a new cannula set loading unit can be taken to a loading position in which the cannula set can be taken from the cartridge and loaded into the cannula insertion system.

If the transfer device cooperates with the storage, the transfer device and/or the transfer device may be arranged to separate a cannula set loading unit from the stack of cannula set loading units.

The transfer device may also be arranged to discharge a cartridge from which the cannula set is introduced into the cannula insertion system. It may also be possible that, after use, the used cannula set is placed back on the cartridge and that the transfer device is arranged to discharge the cartridge and used cannula set. Discharge may comprise placing the used cartridges, with or without used cannula set, back into a stack of cartridges or discharge may be carried out by dropping or placing the used cartridges in a discharge container.

In an embodiment, the transfer device comprises a transfer support to support the cannula set loading unit in a predefined position with respect to the transfer support. By supporting the cannula set loading unit, in particular the cartridge, in a predefined position with respect to the transfer support, the position of the patient needle with respect to the transfer support is also known.

In an embodiment, the transfer support comprises one or more alignment elements to align the cannula set loading unit with respect to the transfer support during loading of the cannula set loading unit on the transfer support. By using alignment element(s), the cannula set loading unit can be easily arranged in the predefined position with respect to the transfer support.

In an alternative embodiment, a sensor, such as a camera or position sensor, may be used to determine the position of the cannula set loading unit with respect to the transfer support.

In an embodiment, the transfer support comprises one or more locking protrusions to releasably lock the cannula set loading unit on the transfer support. By locking the cannula set loading unit, in particular the cartridge, on the transfer support, the cannula set loading unit may be kept in its predefined position with respect to the transfer support. The locking of the cannula set loading unit on the transfer support may also be used to pull a cannula set loading unit from the stack of cannula set loading units.

The locking protrusions of the transfer support may be any locking elements to lock the cannula set loading unit on the transfer support. The cartridge may comprise locking protrusions that cooperate with the locking protrusions on the transfer support to create locking between the cannula set loading unit and the transfer support. The locking protrusions of the cannula set loading unit and/or the transfer support may be spring loaded to facilitate the locking connection between the cannula set loading unit and the transfer support.

In an embodiment, the patient needle comprises a patient needle hub and the cannula set loading device comprises a patient needle hub manipulator arranged to cooperate with the patient needle hub. The patient needle hub manipulator may be any device that can cooperate with the patient needle hub to manipulate the patient needle, for example to couple the patient needle to the cannula insertion device.

In an embodiment, the cannula set comprises a first needle cap, the first needle cap being configured to receive at least a portion (e.g., a distal needle end) of the patient needle, wherein the patient needle hub manipulator is configured to pull the patient needle at least partially out of the first needle cap.

In an embodiment, the cannula set comprises a connection element (e.g., fluid tight connection such as a Luer lock, or a tube collection needle), wherein the patient needle and the connection element are directed in opposite directions. In an embodiment, the connection element is a tube needle arranged to connect the cannula set to a blood collection tube, wherein the connection element hub manipulator is arranged to push the tube needle into a needle holder of a blood collection system of the cannula insertion system. For example, a distal end of the patient needle and a distal end of the connection element may extend in different (e.g., opposite) directions. In some embodiments, the cannula set may lack a tube between the patient needle and the tube needle (or other connection element). For example, the patient needle and the tube needle may extend from a rigid body in different (e.g., opposite) directions. Alternatively, in some embodiments, the cannula set comprises a tube, where, the patient needle is connected a first end of the tube and the connection element is connected at a second end of the tube opposite the first end of the tube to connect the tube to a further device.

In some embodiments, the connection element comprises a connection element hub, wherein the cannula set loading device comprises a connection element hub manipulator arranged to cooperate with the connection element hub. The connection element hub manipulator may be any device that can cooperate with the connection element hub to manipulate the connection element, such as coupling the connection element to a further device to supply or receive fluids.

The invention further provides a cannula insertion system to insert a cannula into a human or animal body, comprising:
a cannula insertion device configured to insert the cannula into the human or animal body,
at least one cannula set loading unit as described herein, and
a cannula set loading device to load a cannula set from the at least one cannula set loading unit into the cannula insertion system.

The cannula insertion system may further comprise:
one or more sensors to determine a suitable location for insertion of the cannula,
a control device arranged to control the positioning system to position, on the basis of the determined location for insertion of the cannula, the cannula insertion device in a suitable position to insert the cannula into human or animal body, and
a blood collection system to collect blood in one or more blood collection tubes supported by the blood collection system.

The blood collection system may comprise a tube needle insertion device to insert the tube needle in the respective blood collection tube and pull the tube needle out of the blood collection tube after the blood collection tube has been filled with blood. The blood collection system may further comprise one or more holding locations to support the one or more blood collection tubes. The holding locations may be arranged in one or more rows, e.g., one or more straight rows or extending along a circular path, that are movable by an actuator with respect to the needle insertion device to align a respective holding location of the holding locations with the needle insertion device to insert the tube needle in the respective holding location.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Further characteristics and advantages of the cannula insertion system of the invention will now be explained by description of an embodiment of the invention, whereby reference is made to the appended drawings, in which:

FIG. 1 shows schematically a cannula insertion system comprising an end-effector supporting a contact sensor and a cannula insertion device;

FIG. 2 shows an isometric view of an example embodiment of a cannula set loading device in accordance with aspects of the present technology;

FIG. 3 shows another isometric view of the cannula set loading device of FIG. 2;

FIGS. 4A-4D show variations of storage for a cannula set loading device in accordance with aspects of the present technology;

FIG. 5 shows an example embodiment of a cannula set loading unit in accordance with aspects of the present technology;

FIG. 6 shows two cannula set loading units of FIG. 5 placed above each other; in accordance with aspects of the present technology; and

FIG. 7 shows the cannula set loading unit of FIG. 4 above a transfer support of the cannula set loading device of FIGS. 2 and 3; and

FIG. 8 shows the cannula set loading unit of FIG. 5 placed on the transfer support of the cannula set loading device of FIGS. 2 and 3;

FIGS. 9A-9E shows schematics of different example embodiments of a cannula set loading unit in accordance with aspects of the present technology;

FIG. 10 shows a collective arrangement of multiple instances of an example embodiment of a cannula set loading unit, in accordance with aspects of the present technology;

FIG. 11A shows an example embodiment of a cannula set loading unit in accordance with aspects of the present technology, and FIG. 11B shows a cartridge of the cannula set loading unit of FIG. 11A;

FIG. 12 shows an example embodiment of storage for a cannula set loading device in accordance with aspects of the present technology;

FIG. 13 shows an example embodiment of a tray for a cannula set in accordance with aspects of the present technology;

FIG. 14 show an example embodiment of a tray for a cannula set in accordance with aspects of the present technology;

FIG. 15 shows an example embodiment of storage for a cannula set loading device in accordance with aspects of the present technology; and

FIG. 16 shows an example embodiment of storage for a cannula set loading device in accordance with aspects of the present technology.

### DETAILED DESCRIPTION

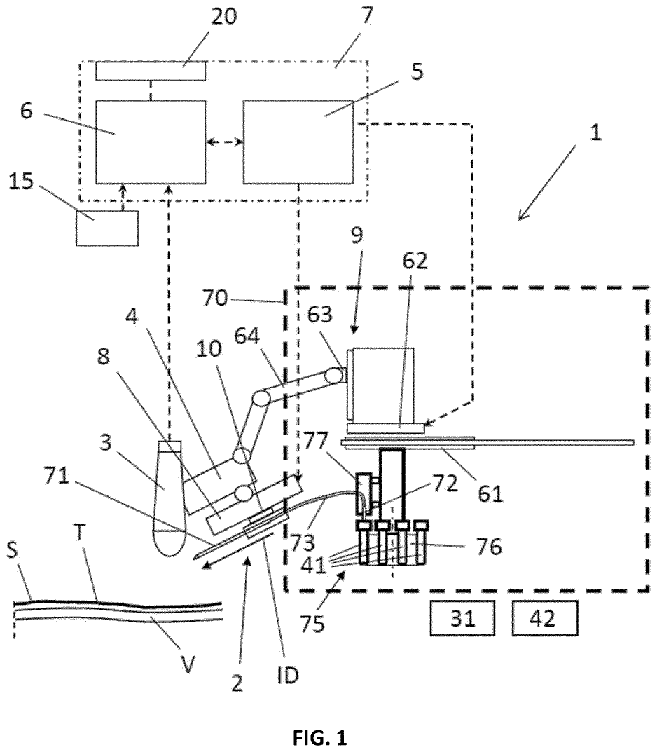

Specific details of several embodiments of the technology are described below with reference to FIGS. 1-16.

### 1. Cannula Insertion System Overview

FIG. 1 shows schematically a cannula insertion system, generally denoted by reference numeral 1.

The cannula insertion system 1 is configured to autonomously insert a patient needle 71 of a cannula set 2 into a blood vessel V of a human or animal. The cannula set 2 comprises the patient needle 71 to be inserted into a patient, a tube needle 72 to be inserted in a blood collection tube 41 and a tube 73. The patient needle 71 is connected at one of the tube 73 and the tube needle 72 is connected at the other opposite end of the tube 73. The cannula set 2 is provided as a disposable part.

The cannula insertion system 1 as shown in FIG. 1 is in particular configured to autonomously draw blood from a blood vessel V. In alternative embodiments, the cannula insertion system 1 may for example be configured to arrange the patient needle 71 in a blood vessel V for intravenous medication and/or infusions.

To draw blood from the blood vessel V, the cannula insertion system 1 may be arranged to determine a location of a blood vessel underneath the skin S of the human or animal, insert a patient needle 71 of a cannula set 2 into the blood vessel V and draw blood from the blood vessel V without direct interaction of an operator of the cannula insertion system 1.

The cannula insertion system 1, shown in FIG. 1, comprises an ultrasound transducer 3 to obtain one or more sensor signals that are representative for the location of a blood vessel V in the human or animal. The ultrasound transducer 3 is a contact sensor. During use contact between the ultrasound transducer 3 and the skin S is required in order to obtain relevant data with respect to the location of the blood vessel V. The ultrasound transducer 3 may be guided along the skin S of the human or animal in a target area T. This target area T is an area of the skin S underneath which the presence of a blood vessel V suitable for insertion of a cannula set 2 is expected and which is examined by the ultrasound transducer 3. A control device 5 controls the position of the ultrasound transducer 3.

The target area T may be determined by obtaining images of the skin of a human or animal, for example using NIR (near infrared), infrared or visible light sensors 15, and determining on the basis of the images an area in which it is likely that a blood vessel suitable for insertion of a cannula will be found.

Once a target area T is determined, the ultrasound transducer 3 may be used to provide a sensor signal representative for a location of the blood vessel V within the target area T. The sensor signal is fed into a processing device 6 which is arranged to process the sensor signal. The control device 5 and the processing device 6 may be comprised in a single processor 7, such as a PC, or may be comprised in multiple processors (co-located or distributed).

On the basis of the sensor signal provided by the ultrasound transducer 3, an image of the vein may be created by the processing device 6. This image may be based on multiple 2D images along a line in a certain direction, 3D coordinates and direction, 6D coordinates or may be a 3D image.

The processing device 6 determines on the basis of the sensor signal, or sensor signals, the location of a blood vessel V suitable for the insertion of a patient needle 71 of the cannula set 2. On the basis of this location, the processing device 6 may determine an insertion path for insertion of the patient needle 71 of the cannula set 2 into the blood vessel V.

The patient needle 71 is releasably held by a cannula holder 10 of a cannula insertion device 8. The patient needle 71 can be released from the cannula holder 10 to facilitate exchange of cannula sets 2.

The cannula insertion device 8 is arranged to insert the patient needle 71 in an insertion direction ID through the skin S and into the blood vessel V along the insertion path determined by the processing device 6.

The cannula insertion device 8 is supported by a positioning system 9, for example a robot arm, that is arranged to bring the cannula insertion device 8 in a position from which the cannula insertion device 8 may move the patient needle 71 along the insertion path. The cannula insertion device 8 and the positioning system 9 are controlled by the control device 5.

In the embodiment shown in FIG. 1, the ultrasound transducer 3 and the cannula insertion device 8 are mounted on a single end-effector 4. This ensures a fixed spatial relationship between the ultrasound transducer 3 and the cannula insertion device 8. The positioning system 9 is therefore used for positioning of both the ultrasound transducer 3 and the cannula insertion device 8.

In the shown embodiment of FIG. 1, the positioning system 9 for positioning of the end-effector 4 comprises a first stage 61 to move the end-effector 4 in x-direction, a second stage 62 to move the end-effector 4 in y-direction and a third stage 63 to move the end-effector in z-direction, wherein the x-direction, y-direction and z-direction are orthogonal directions. The positioning system 9 further comprising a connection arm 64 comprising three rotation actuators to rotate the end-effector 4 about each of the x-direction, y-direction and z-direction.

This results in end-effector 4 being movable in six degrees of freedom to optimally position the end-effector 4 with respect to a human or animal body, for example a patient's arm.

The first stage 61 has a relatively long stroke that can be used to move the end-effector 4 between an operating position (shown in FIG. 1) from which a patient needle 71 of the cannula set 2 may be inserted into the human or animal body, and a retracted position. In dashed lines a housing 70 of the cannula insertion system 1 is shown. This housing 70 comprises a recess in which the cannula insertion device 8 may at least partially be placed. During the insertion of the patient needle 71 into a patient, the first stage 61 may be arranged in the operating position in which the end-effector 4 extends at least partially outside the housing 70. After blood withdrawal is finished, the end-effector 4 including the cannula insertion device 8 may be moved to the retracted position so that the cannula insertion device 8 no longer extends from the housing 70. In the retracted position, within the housing 70, the cannula insertion system 1 may exchange disposable parts such as a cannula and blood collection tubes filled during blood withdrawal. Also, new disposable parts may be provided to facilitate blood withdrawal of a subsequent patient. For example, in the retracted position of the end-effector 4, the cannula set 2 may be exchanged at a cannula set loading device 31 and the blood collection tubes 41 may be exchanged at blood collection tube exchange device 42.

To fill a blood collection tube 41 with blood the tube needle 72 is inserted into the blood collection tube 41 and the patient needle 71 into a blood vessel of the human or animal body. Advantageously, the tube 73 between the patient needle 71 and the tube needle 72 can be relatively short so that a relatively low volume of blood is needed to fill the conduit 73. For this reason, the blood collection tubes 41 are held by a blood collection system 75 which is mounted on the first stage 61 of the positioning system 9. As a result, the blood collection system 75 will move together with the end-effector 4 between the operating position and the retracted position and will stay relatively close to the end-effector 4.

The blood collection system 75 comprises a carousel 76 comprising multiple holding locations each arranged to hold a blood collection tube 41 and a tube needle insertion device 77 to insert the tube needle 72 in a blood collection tube 41 aligned with the tube needle insertion device 77 and pull the tube needle 72 out of the blood collection tube 41 after the blood collection tube 41 has been filled with blood. The carousel 76 is rotatable about an axis of rotation extending in z-direction. By rotation about the axis of rotation a selected one of the holding locations can be aligned with the tube needle insertion device 77 to allow the tube needle 72 to be inserted into the blood collection tube held in the selected one of the holding locations. When the blood collection tube 41 is filled, the tube needle insertion device 77 may retract the tube needle 72 out of the blood collection tube 41 and a new holding location and blood collection tube 41 held therein may be aligned with the tube needle insertion device 77 by rotation of the carousel 76. When arranged in this position the tube needle insertion device 77 may insert the tube needle 72 into this new blood collection tube 41. To determine whether a blood collection tube is sufficiently filled a sensor may be provided that is capable of providing a level signal representative for sufficient filling of the blood collection tube 41. This sensor is for example a level sensor that can measure whether a predetermined level of blood in the blood collection tube 41 has been reached, for example by using a light beam.

### 2. Cannula Set Loading Device

In some embodiments, a system may include a cannula set loading device 31 that functions to load a cannula set to a desired position in or relative to the cannula insertion system, such as to a desired position in relation to a cannula insertion device 8 that can insert a patient needle from the loaded cannula set into a patient body. For example, a cannula set loading device can load a cannula set loading unit that carries a cannula set.

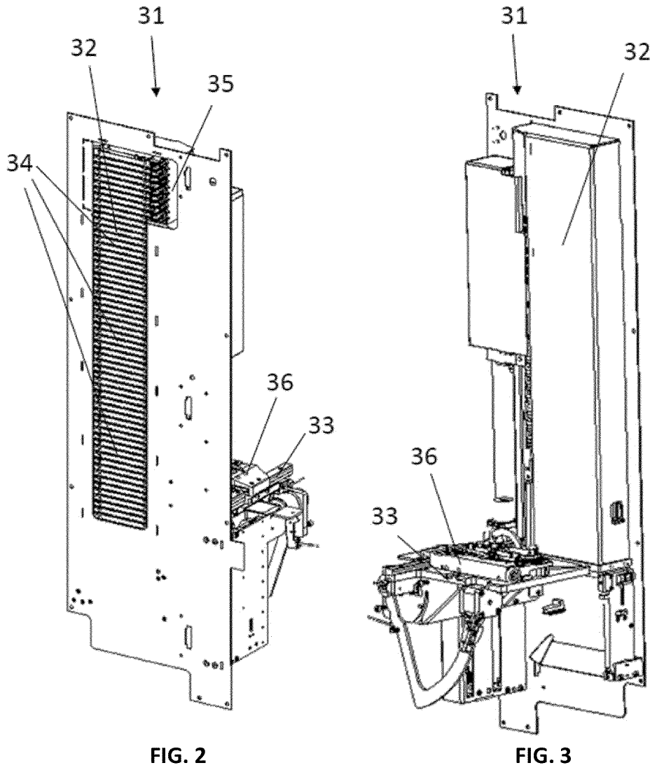

FIGS. 2 and 3 show in more detail an example embodiment of a cannula set loading device 31 to load a cannula set 2 into the cannula insertion system 1.

The cannula set loading device 31 comprises a storage 32 and a transfer device 33. The storage device 32 is configured to hold a stack of cannula set loading units 34 in a collective arrangement, wherein each cannula set loading unit 34 carries a cannula set 2. The storage 32 comprises a storage opening 35 to introduce the cannula set loading units 34 into the storage 32. The storage opening 35 may be accessible from the exterior of the housing 70 of the cannula insertion system 1.

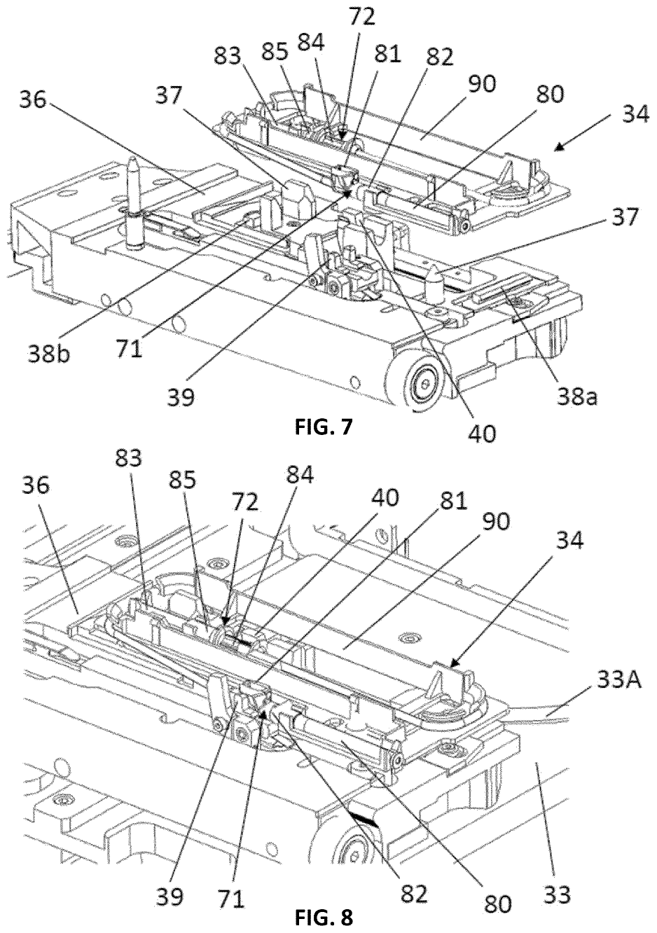

The transfer device 33 is arranged to transfer a cannula set loading unit 34 from a storage position in the storage 32 to a loading position. In the loading position, the cannula set 2 may be taken from the cannula set loading unit 34 and introduced into the cannula insertion system 1. The transfer device 33 comprises a transfer support 36 to support the cannula set loading unit 34 in a predefined position with respect to the transfer support 36. In the storage position, a cannula set loading unit 34 can be taken from the stack of cannula set loading units 34 and placed on the transfer support 36. In the shown embodiment, the bottom cannula set loading unit 34 is taken from the bottom end of the stack of cannula set loading units 34 for use with the cannula insertion system 1. For example, in some embodiments a biasing element (e.g., plate supported by a spring) can be placed laterally adjacent to the bottom cannula set loading unit 34 to passively urge the bottom cannula set loading unit 34 from its storage position to the loading position away from (e.g., misaligned with) the remaining stacked cannula loading units 34. Additionally or alternatively, an actuatable arm can actively laterally push or pull the bottom cannula set loading unit 34 from the remaining stacked cannula set loading units 34. An illustrative example of operation of the cannula set loading device is described below with reference to FIGS. 7 and 8.

In some embodiments, a cannula set loading unit 34 can be selected and extracted from any suitable location(s) in the stack of cannula set loading units 34 for use with the cannula insertion system 1, such as a first end of the stack, a second end of the stack, or any intermediate location between the first and second ends of the stack. For example, in some embodiments an uppermost cannula set loading unit 34 can be taken from the upper end of the stack of cannula set loading units 34. As another example, an intermediate cannula set loading unit 34 located between the bottom and upper ends of the stack of cannula set loading units 34.

With respect to the embodiment shown in FIGS. 2 and 3, when the bottom cannula loading 34 is extracted from the stack of cannula loading units 34, gravity may induce the remaining cannula loading units to shift downwards, thereby positioning another cannula set loading unit 34 into a location suitable for transfer to the loading position. In some embodiments, the cannula set loading device 31 may additionally or alternatively include a biasing element (e.g., plate supported by a spring) placed against either or both ends of the stack (e.g., the bottom end and/or upper end of the stack of cannula set loading units 34) to help position another cannula set loading unit 34 into a location suitable for transfer to the loading position and/or help maintain the compactness of the stack of cannula set loading units 34.

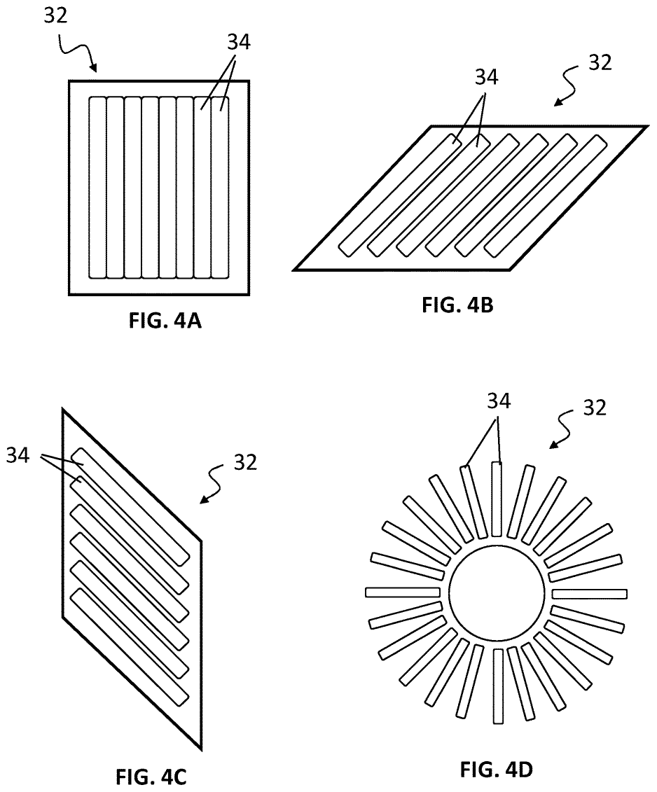

Although FIGS. 2 and 3 illustrate the storage 32 as storing multiple cannula set loading units 34 arranged adjacent to one another in a vertical stack, it should be understood that the cannula set loading device 31 can store a collection of multiple cannula set loading units 34 in any suitable arrangement. For example, FIG. 4A illustrates multiple cannula set loading units 34 arranged adjacent to one another in a horizontal stack (e.g., in a row). In some embodiments, the storage 32 can store multiple cannula set loading units 34 positioned at an angle, such as in a horizontal angled stack (FIG. 4B) or a vertical angled stack (FIG. 4C). As another example, the storage 32 can store multiple cannula set loading units 34 in a vertical or horizontal carousel arrangement, such as a circular carousel arrangement shown in FIG. 3D. In these and various suitable embodiments, an individual cannula set loading unit 34 can be extracted from its storage position in the storage 32 and transferred to a loading position (e.g., with a transfer device 33), similar to that described above with respect to FIGS. 2 and 3.

It should be understood that the specific number of cannula set loading units 34 shown in FIGS. 2-4D are for illustrative purposes only, and the storage 32 can be sized to store any suitable number of cannula set loading units 34, such as at least fifty, at least one hundred, at least two hundred, or more.

### 3. Cannula Set Loading Unit

As described herein, a cannula set loading unit 34 includes a cannula set 2 and a cartridge 90 configured to hold at least a portion of the cannula set 2 in a predetermined position or configuration relative to the cartridge 90. For example, in some embodiments, the cannula set 2 can include a patient needle 71, a tube needle 72, and a tube 73 extending between the patient needle 71 and the tube needle 72 for enabling fluidic communication between the patient needle 71 and the tube needle 72. In such embodiments, the cartridge 90 may include one or more fixation elements (e.g., snap connectors, recesses, etc.) configured to hold the patient needle 71, the tube needle 72, and/or the tub 73 in a predetermined or fixed position or configuration.

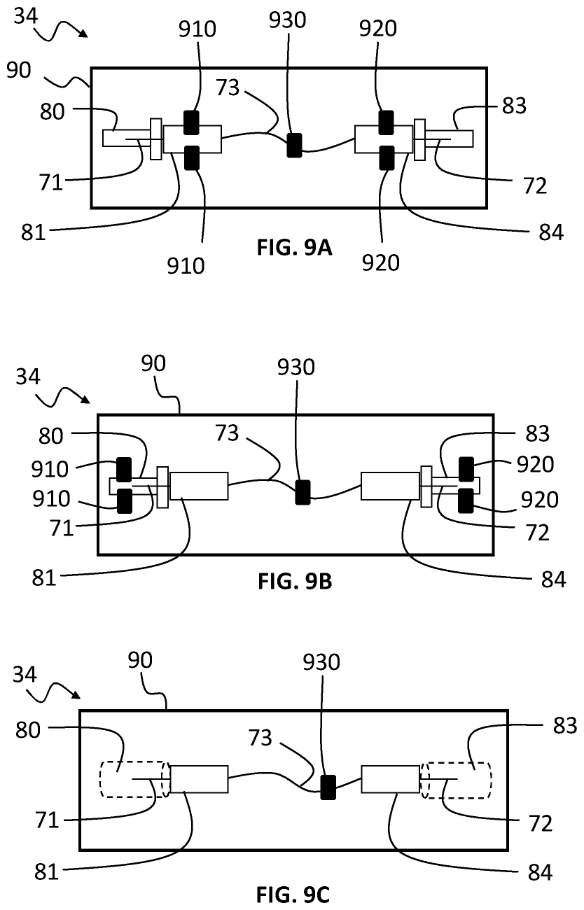

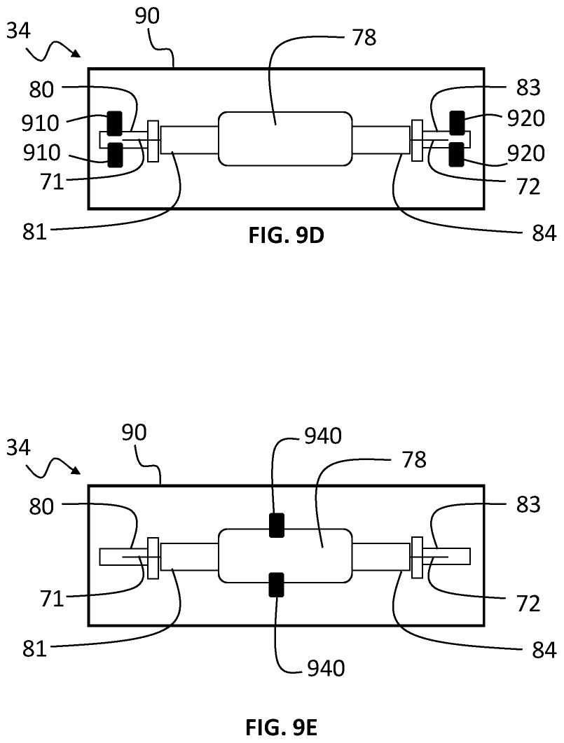

The fixation elements of the cartridge 90 may be configured to removably couple to feature(s) of the cannula set 2 in any of various suitable manners, to hold one or more portions of the cannula set 2 in a predetermined position or configuration with respect to the cartridge 90. For example, FIG. 9A is a schematic illustration of a cannula set loading unit 34 configured to hold a cannula set that includes a patient needle 71 including a patient needle hub 81, a tube needle 72 including a tube needle hub 84, and a tube 73 fluidically connecting the patient needle 71 and the tube needle 72. The cannula set also includes a first needle cap 80 for receiving and covering the patient needle 71, and a second needle cap 83 for covering the tube needle 72. In some embodiments, the patient needle 7 can be oriented in a fixed rotational orientation relative to the first needle cap 80, and the tube needle 72 can be oriented in a fixed rotational orientation relative to the second needle cap 83. In the example cannula set loading unit 34 shown in FIG. 9A, the cartridge 90 includes one or more fixation elements 910 for securing the patient needle 71 in a first fixed location and/or orientation and one or more fixation elements 920 for securing the tube needle 72 in a second fixed location and/or orientation. In particular, the fixation element(s) 910 are configured to removably couple to the patient needle hub 81 for securing the patient needle 71, and the fixation element(s) 920 are configured to removably couple to the tube needle hub 84. Furthermore, the cartridge 90 can include one or more fixation elements 930 configured to removably couple to and hold the tube 73 in a fixed location and/or orientation.

As another example, FIG. 9B is a schematic illustration of a cannula set loading unit 34 configured to hold a cannula set that is similar to the cannula set described above with respect to FIG. 9A. As described above, the patient needle 7 can be oriented in a fixed rotational orientation relative to the first needle cap 80, and the tube needle 72 can be oriented in a fixed rotational orientation relative to the second needle cap 83. However, in the example cannula set loading unit 34 shown in FIG. 9B, the fixation element(s) 910 are configured to removably couple to the first needle cap 80 (thereby indirectly securing the patient needle 71 to the cartridge 90 in a fixed location and/or orientation with respect to the cartridge), and the fixation element(s) 920 are configured to removably couple to the second needle cap 83 (thereby indirectly securing the tube needle 72 to the cartridge 90 in a fixed location and/or orientation with respect to the cartridge). Like the cartridge 90 shown in FIG. 9A, the cartridge 90 shown in FIG. 9B may, in some embodiments, further include one or more fixation elements 930 configured to removably couple to and hold the tube 73 in a fixed location and/or orientation.

As another example, FIG. 9C is a schematic illustration of a cannula set loading unit 34 configured to hold a cannula set that includes a patient needle 71 including a patient needle hub 81, a tube needle 72 including a tube needle hub 84, and a tube 73 fluidically connecting the patient needle 71 and the tube needle 72. In the cannula set loading unit 34 shown in FIG. 9C, the cartridge 90 can include one or more integrated features functioning as needle caps for the patient needle 71 and the tube needle 72. For example, the cartridge 90 can include a first recess (e.g., blind hole) functioning as a first needle cap 80 that receives at least a portion of the patient needle 71, and/or a second recess (e.g., blind hole) functioning as a second needle cap 83 that receives at least a portion of the tube needle 72. Additionally or alternatively, the first needle cap 80 and/or the second needle cap 83 can be formed separately from the cartridge 90, and fixedly coupled to the cartridge 90. In some embodiments, the patient needle 7 can be oriented in a fixed rotational orientation relative to the first needle cap 80, and the tube needle 72 can be oriented in a fixed rotational orientation relative to the second needle cap 83. Accordingly, the first and second needle caps 80 and 83 that are integrated in or coupled to the cartridge 90 can hold the patient needle 71 and the tube needle 72 in a fixed location and/or orientation with respect to the cartridge. Like the cartridge 90 shown in FIG. 9A, the cartridge 90 shown in FIG. 9C may, in some embodiments, further include one or more fixation elements 930 configured to removably couple to and hold the tube 73 in a fixed location and/or orientation.

In some embodiments a cannula set 2 may lack a tube 73. For example, as shown in the schematics of FIGS. 9D and 9E, a cannula set may include a patient needle 71 with a patient needle hub 81 and a tube needle 72 coupled to a tube needle hub 84. The patient needle hub 81 (or the patient needle 71 itself) and the tube needle hub 84 (or the tube needle 72 itself) are each coupled to different portions of a rigid member 78 that allows fluidic communication between the patient needle 71 and the tube needle 72. Any of the cartridges 90 described herein can be modified to secure such a tubeless cannula set 2. For example, FIG. 9D is a schematic illustration of a cannula set loading unit 45 including a cartridge 90 similar to the cartridge 90 described above with respect to FIG. 9B, with fixation element(s) 910 configured to couple to the first needle cap 80 and fixation element(s) 920 configured to couple to the second needle cap 83. Similarly, the cartridge 90 shown in FIG. 9A (with fixation element(s) 910 configured to couple to the patient needle hub 81 and fixation element(s) 920 configured to couple to the tube needle hub 84) can be modified to secure a tubeless cannula set 2. Furthermore, the cartridge 90 shown in FIG. 9C (with needle caps 80 and 83 that are integrated in or coupled to the cartridge 90) can be modified to secure a tubeless cannula set 2.

In some embodiments, a tubeless cannula set includes a generally rigid body or assembly, such that it can be secured to a cartridge 90 at relatively few locations (e.g., one or two) sufficient for securing the tubeless cannula set in a fixed configuration with a known location and/or orientation. For example, as shown in FIG. 9E, a cartridge 90 can include one or more fixation elements 940 configured to removably couple to and hold the rigid member 78 to the cartridge in a fixed location and/or orientation, thereby holding the patient needle 71 and/or the tube needle 72 in a fixed location and/or orientation by virtue of their fixed relationship to the rigid member 78.

It should also be understood that in other embodiments, a cannula set loading unit 34 can include any combination of cartridge features shown in and described above with respect to FIGS. 9A-9E. For example, a cartridge 90 can include fixation elements 910 configured to removably couple to the patient needle hub 81 as well as the first needle cap 80, and/or fixation elements 920 configured to removably couple to the tube needle hub 84 as well as the second needle cap 83.

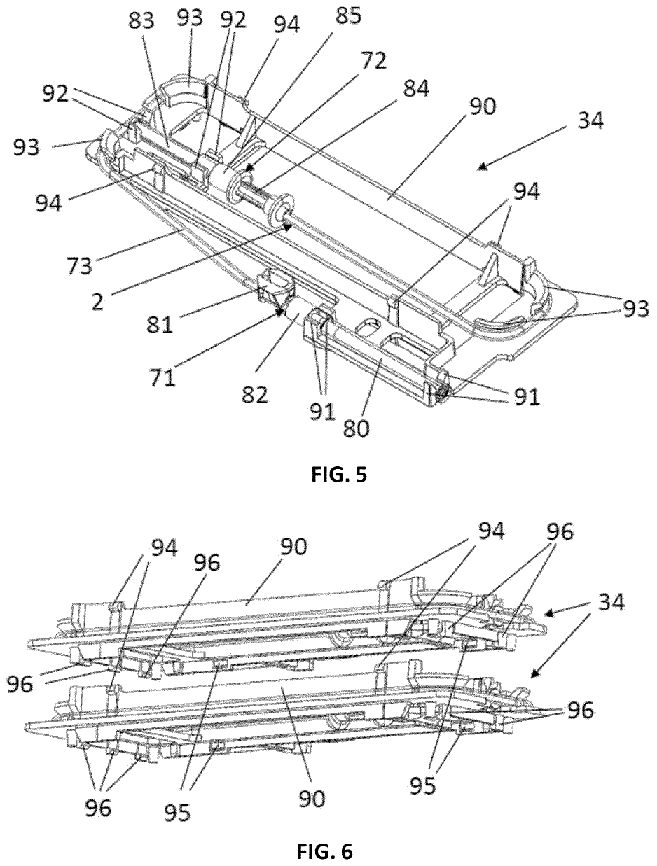

FIG. 5 shows an example embodiment of a cannula set loading unit 34 in more detail. Various features (e.g., snap connections) described herein with respect to this example embodiment may alternatively be applied to any of the cartridge configurations shown in and described above with respect to FIGS. 9A-9E. The cannula set loading unit 34 comprises a cartridge 90 having predefined locations for holding at least a portion of the cannula set 2 in a fixed position with respect to the cartridge 90. The cannula set 2 shown in FIG. 5 comprises patient needle 71, tube needle 72 and tube 73 extending between the patient needle 71 and the tube needle 72 for enabling fluidic communication between the patient needle 71 and the tube needle 72.

As shown in FIG. 5, the patient needle 71 comprises a first needle cap 80 and a patient needle hub 81. The first needle cap 80 is provided to accommodate the needle end of the patient needle 71 to help ensure that the needle end is not contaminated before use. The patient needle 71 may be configured to hold the needle end and the patient needle hub 81 in a fixed rotational position with respect to the first needle cap 80. A paper ring 82 is arranged between the first needle cap 80 and the patient needle hub 81 as a tamper indicator. This tamper indicator may be used to check whether the first needle cap 81 was arranged properly on the needle end before the patient needle is used. The paper ring may also ensure that the rotational position between the first needle cap 80 and the patient needle hub 81 is fixed. The patient needle hub 81 is provided to allow manipulation of the patient needle 71, such as without directly touching the patient needle 71.

The upper side of the patient needle hub 81 is designed to be releasably connected to the cannula holder 10 of the cannula insertion device 8. The patient needle hub 81 may also be used to pull the needle end of the patient needle 71 out of the first needle cap 80.

The cartridge 90 comprises snap connection elements 91 to hold the first needle cap 80 by a snap connection on the cartridge 90. The surfaces of the snap connection elements 91 and the first needle cap 80 are designed to hold the first needle cap 80 in a fixed position and orientation with respect to the cartridge 90 such that the location of the patient needle hub 81 is also known. This predefined position and orientation of the patient needle 71 on the cartridge 90 can effectively be used to couple and manipulate the patient needle 71, for instance in order to couple the patient needle 71 to the cannula holder 10.

The tube needle 72 comprises a second needle cap 83 and a tube needle hub 84. The second needle cap 83 is configured to maintain the needle end of the tube needle 72 that is to be inserted into a blood collection tube 41 in a closed, sterile environment. A paper ring 85 is placed as a tamper evident between the second needle cap 83 and the tube needle hub 84. The tube needle hub 84 can be used to manipulate the tube needle 72, for example to pull the needle end of the tube needle 72 out of the second needle cap 83 and/or to couple the tube needle 72 to a tube needle insertion device 77 of a blood collection system 75.

The cartridge 90 comprises snap connection elements 92 to hold the second needle cap 83 by a snap connection on the cartridge 90. The surfaces of the snap connection elements 92 and the second needle cap 83 are designed to hold the second needle cap 83 in a fixed position and orientation with respect to the cartridge 90 such that the location of the patient needle hub 81 with respect to the cartridge 90 is also defined. This predefined location of the tube needle 72 on the cartridge 90 can effectively be used to manipulate the tube needle 72.

The cartridge 90 comprises tube holding elements 93 to hold the tube in a loop on the cartridge 90. For example, as shown in FIG. 5, a tube holding element 93 can include a curved cradle (e.g., concave wall that engages the outer surface of the tube 73) that routes at least a portion of the tube in a curving arrangement. In some alternative embodiments, a tube holding element 93 can include a post around which the tube 73 can wrap. As shown in FIG. 5, the multiple tube holding elements 93 are arranged to route the tube 73 generally in a spiraling manner (e.g., the patient needle 71 is in an outer turn of the spiral and the tube needle 72 is in an inner turn of the spiral, or vice versa). The tube holding elements 93 are arranged such that the tube 73 will be held by the tube holding elements 93 as long as the patient needle 71 and the tube needle 72 are held in the predefined locations on the cartridge 90 as shown in FIG. 4. When the patient needle 71 is pulled out of the first needle cap 80 and/or the tube needle 72 is pulled out of the second needle cap 83, the tube 73 may also be released from its fixed position on the cartridge 90 due to the loop shape with which the tube is held on the cartridge 90.

The cartridge 90 comprises snap connection elements to snap the cartridge 90 on a second cartridge 90 in order to form a stack of cannula set loading units 34 that can be loaded as a single package of for example five or ten cannula set loading units 34 in the storage 32 of the cannula loading device 31.

FIG. 6 shows two cartridges with cannula sets 2 mounted thereon above each other. The snap connection elements to connect the cartridges 90 comprise snap protrusions 94 at one side of the cartridge 90 and snap recesses 95 on the opposite side of the cartridge 90. The snap protrusions 94 can be arranged in the snap recesses 95 to create the snap connections between stacked cartridges 90. These snap connections can help enable removable coupling between adjacent cartridges. In other embodiments, cartridges may additionally or alternatively be removably attached to one another with other suitable mating or interlocking features, such as a tongue(s) and groove(s) that interlock in a sliding manner, or threads that interlock in a rotational manner. Additionally or alternatively, two or more cartridges may removably couple to one another with one or more suitable fasteners, such as magnets, removable pins, hook and loop fasteners, etc.

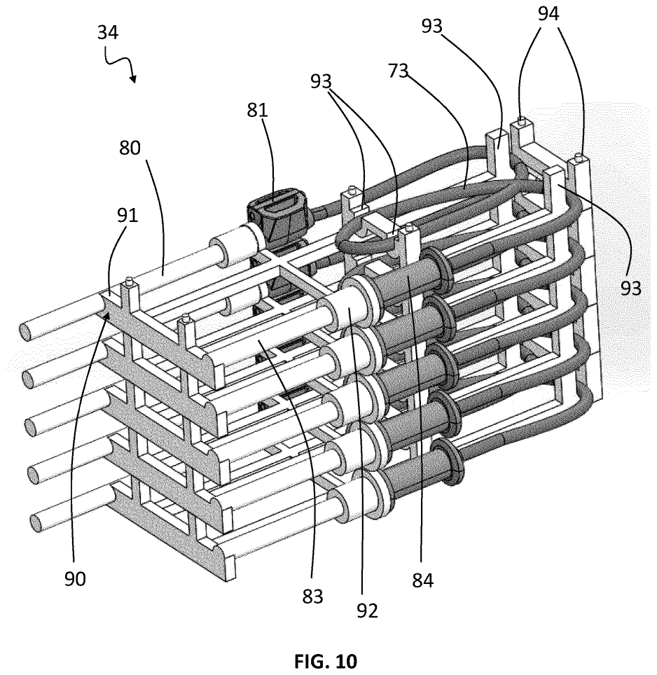

FIG. 10 shows an example embodiment of a cannula set loading unit 34 that includes a cartridge 90 in the form of a frame. The cartridge 90 of FIG. 10 can be similar to the cartridge 90 shown in FIG. 5, except that the cartridge 90 of FIG. 10 does not include a planar base that supports the various securing features of the cartridge (e.g., tube holding elements), and the cartridge 90 of FIG. 10 does not include snap connection elements for engaging the first and second needle caps. Rather, the cartridge 90 of FIG. 10 includes a skeleton frame (e.g., lattice) with interconnected members that support the various securing features of the cartridge 90. Additionally, the cartridge 90 of FIG. 10 includes a first needle cap 80 and a second needle cap 83 that are integrated with the cartridge (e.g., integrally formed with one or more interconnected members of the skeleton frame). However, in some embodiments, the skeleton frame can support snap connection elements similar to snap connection elements 91 to hold a separate first needle cap 80 and/or snap connection elements 92 to hold a separate second needle cap 83 as described above with respect to cartridge 90 shown in FIG. 5. The cartridge 90 can furthermore include one or more tube holding elements 93 to hold the tube 73 in in a loop on the cartridge 90. As shown in FIG. 10, the tube holding element 93 can include, for example, a post around which the tube 73 can wrap, or a snap connection element (e.g., clip that engages with the outer surface of the tube 73). Additionally or alternatively, a tube holding element 93 can include a curved cradle that engages the outer surface of the tube 73, similar to the tube holding elements 93 shown in FIG. 5. Furthermore, like the cartridges 90 shown in FIG. 6, the cartridge 90 of FIG. 10 can include snap connection elements (e.g., snap protrusions 94) to connect multiple cartridge 90 together in a stack.

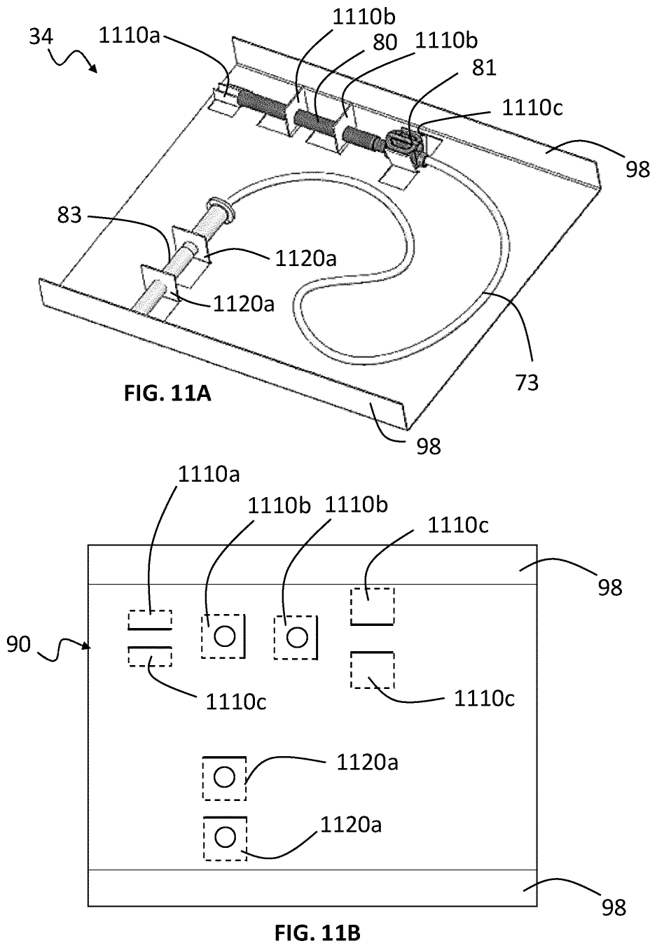

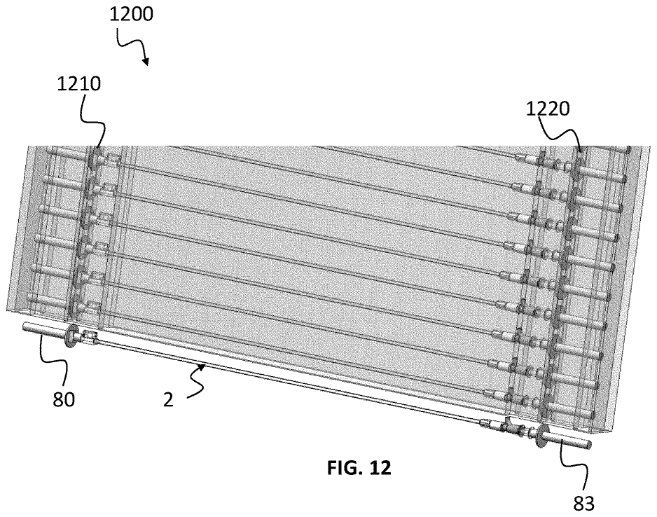

FIG. 11A is a schematic illustration of an example embodiment of a cannula set loading unit 34 including a cartridge 90 constructed from a foldable material (e.g., paper, cardboard, malleable or flexible plastic, etc.). The cartridge 90 can include, for example, one or more flaps for receiving and/or cradling at least a portion of the cannula set, where the flaps are formed from a sheet of foldable material, as shown for example in FIG. 11B. For example, a flap can be formed by cutting (e.g., laser cutting) a perimeter shape of the flap in a base surface of the sheet while maintaining a hinging edge of the flap in the sheet material, then folding the flap to create a three-dimensional feature for engaging the cannula set. In some variations, multiple flaps can be formed in a cradling arrangement (e.g., two opposing flaps) configured to receive and secure a portion of the cannula set. Additionally or alternatively, in some variations a flap can include a hole for receiving a portion of the cannula set therethrough. In some embodiments, the cartridge 90 can further include one or more sidewalls 98, formed by folding an edge of the sheet of the cartridge material (e.g., orthogonally).

In the example embodiment shown in FIG. 11A, the cartridge 90 includes a first series of flaps configured to engage the first needle cap 80 covering the patient needle of the cannula set, a second series of flaps configured to engage the patient needle hub 81 of the cannula set, and a third series of flaps configured to engage the second needle cap covering the tube needle of the cannula set. The first series of flaps includes two opposing cradling flaps 1110a configured to receive an end of the first needle cap 80 therebetween, as well as multiple flaps 1110b each having a circular hole formed in each of their faces for receiving the first needle cap 80 therethrough. The second series of flaps includes two opposing cradling flaps 1110b configured to receive the patient needle hub 81 therebetween. The third series of flaps includes multiple flaps 1120a configured to each having a circular hole formed in each of their faces for receiving the second needle cap 83 therethrough. In some embodiments, the cartridge 90 can include fewer or more series of flaps, such as a fourth series of flaps (not shown) configured to secure a segment of the tube 73. Furthermore, it should be understood that while the example shown in FIGS. 11A and 11B include multiple series of flaps arranged relative to one another in a certain configuration, the multiple series of flaps can be arranged in any suitable manner relative to one another for positioning the patient needle, the tube needle, tube, and/or any other components of the cannula set in predetermined location(s) with respect to the cartridge 90.

Similar to other embodiments of the cartridge 90 described herein, multiple instances of the cartridge 90 shown in FIG. 11A can be stackable. For example, the sidewalls 98 can be of sufficient height (e.g., greater than the height of the cannula set on the cartridge 90) to offset an adjacent cartridge 90 with enough spacing so as to provide clearance for the cannula set arranged on the cartridge 90 between the sidewalls 98. Although the example embodiment shown in FIGS. 11A and 11B include two opposing sidewalls, it should be understood that the cartridge can have any suitable number of side walls (e.g., one, two, three, four, or more). In some embodiments, the sidewalls 98 can be reinforced to help maintain spacing between adjacent stacked cartridges 90. For example, the sidewalls 98 can include thicker material resistant to buckling (e.g., the cartridge sheet material can be backed by a reinforcing second sheet of material).

### 4. Method of Cannula Set Loading

Generally, a cannula set loading unit 34 can be automatically removed from a stack of cannula set loading units 34 and loaded in a known position and/or orientation relative to one or more components of a cannula insertion system 1. Various components of the loaded cannula set loading unit 34 can be automatically removed from the cannula set loading unit 34, such as the patient needle 71, the tube needle 72, and/or the tube 73 extending between the patient needle 71 and the tube needle 72 (if present in the cannula set). During this process, the patient needle 71 can be automatically separated from the first needle cap 80 (which can disrupt a first tamper indicator arranged therebetween) and/or the tube needle 72 can be separated from the second needle cap 83 (which can disrupt a second tamper indicator arranged therebetween). The tube needle can be automatically coupled to a blood collection tube or other further device for blood collection, and/or the patient needle 71 can be automatically inserted into a patient (e.g., human or animal body). Accordingly, the cannula set from the cannula set loading unit 34 can be automatically positioned so as to allow fluid communication from and/or to the patient and the blood collection tube (or other further device).

An illustrative method of cannula set loading is described below with reference to FIGS. 7 and 8. FIG. 7 shows the cannula set loading unit 34 shown in FIG. 5 arranged above the transfer support 36 of the transfer device 33 shown in FIGS. 2 and 3. FIG. 8 shows the cannula set loading unit 34 loaded on the transfer support 36. The transfer support 36 comprises alignment elements 37 to align the cannula set loading unit 34 with respect to the transfer support 36 during loading of the cannula set loading unit 34 on the transfer support 36. The alignment elements 37 have slanted top surfaces that will guide the cartridge 90 to a predefined position on the transfer support 36.

The transfer support 36 further comprises first locking protrusions 38a and second locking protrusions 38b to releasably lock the cannula set loading unit 34, in particular the cartridge 90, on the transfer support 36. The first and second locking protrusions 38a, 38b cooperate with locking elements 96 on the cartridge 90 (see FIG. 6) to obtain a locking connection between the cartridge 90 and the transfer support 36. At least one of the first and second locking protrusions 38a, 38b is spring loaded and biased away from the other of the second and first locking protrusions 38b, 38a, respectively. The slanted surfaces on the first and second locking protrusions 38a, 38b allow the spring loaded first and/or second locking protrusions 38a, 38b to be pushed by the locking elements 96 against the spring force so that the locking elements 96 may engage with the first and second locking protrusions 38a, 38b. This engagement can be used to pull a cannula set loading unit 34 from the bottom of the stack of cannula set loading units 34 in the storage 32 and/or to maintain a fixed position of the cartridge 90 with respect to the transfer support 36.

The transfer device 33 further comprises a patient needle hub manipulator 39 arranged to cooperate with the patient needle hub 81. The patient needle hub manipulator 39 may engage the patient needle hub 81 to pull the needle end of the patient needle 71 at least partly out of the first needle cap 80. This movement of the patient needle 71 with respect to the first needle cap 80 may also destruct the tamper evident 82 provided between the patient needle 71 and the first needle cap 80.

The patient needle hub 81 may be directly connected to the cannula holder 10 of the cannula insertion device 8. Once connected to the cannula holder 10, the positioning system 9 of the cannula insertion device 8 may also be used to pull the needle end of the patient needle 71 out of the first needle cap 80.

Correspondingly, transfer device 33 comprises a tube needle hub manipulator 40 arranged to cooperate with the tube needle hub 84. The tube needle hub manipulator 40 may engage the tube needle hub 84 to pull the needle end of the tube needle 72 at least partly out of the second needle cap 83. This movement of the tube needle 72 with respect to the second needle cap 83 may also destruct the tamper evident 85 provided between the tube needle 72 and the second needle cap 83. A slot 33A in a stationary surface of the transfer device 33 may be used to induce movement of the tube needle hub manipulator 40 to pull the needle out of the second needle cap 83 when the transfer support 36 is moved from the storage position to the loading position.

Movement of the patient needle 71 out of the first needle cap 80 and/or of the tube needle 72 out of the second needle cap 83 will also release the tube 32 from the tube holding elements 93. As a result, the cannula set 2 may be taken from the cartridge 90.

The tube needle hub manipulator 40 may be used to move the tube needle 72 towards and into a needle holder of the tube needle insertion device 77 of the blood collection system 75 of the cannula insertion system 1.

After the cannula set 2, excluding the first needle cap 80 and the second needle cap 83, is taken from the cartridge 90 and load into the cannula insertion system 1, the cartridge 90 together with the first and second needle cap 80, 83 fixed to the cartridge 90 by snap connection elements 91, 92, may be discarded. This may be carried out by the cannula set loading device 31 or a separate device. Discarding the cartridge 90 may be carried out in the storage position, loading position or any other suitable position. The discarded cartridges 90 may be collected in a container. The cartridges 90 may be disposable or reusable, for example after sterilisation. An operator of the cannula insertion system (or component thereof, such as the cannula set loading device 31 itself) can load each cartridge 90 with a respective cannula set to form cannula set loading unit(s) 34 that are stocked and ready for use with the cannula insertion system. A cartridge-loading process can be performed for cartridges 90 that are disposable (e.g., single one-time insertion of a cannula set onto a single-use, disposable cartridge 90), or reusable (e.g., multiple, repeated insertion of a cannula set onto a reusable cartridge 90). In some embodiments, during a cartridge-loading process, a cartridge 90 can be outside of (e.g., removed from, separated from) the cannula set loading device 31. Additionally or alternatively, during a cartridge-loading process, a cartridge 90 can be retained in or with the cannula set loading device 31. For example, one or more cartridges 90 can be fixedly coupled to, removably coupled to, or integrated within the cannula set loading device 31, and accessed by an operator for loading with a cannula set. As an illustrative example embodiment, the one or more cartridges 90 can be accessed through a door or other opening, or can be accessed in a cannula set loading device 31 that is extendable outside a housing of the cannula insertion system.

When the cartridge 90 is discarded, the transfer device 33 may collect a new cannula set loading unit 34 from the bottom of the stack of cannula set loading units 34 in the storage 32.

### 5. Other Example Cannula Set Loading Arrangements

Other examples of cannula set loading arrangements are described below with reference to FIGS. 12-16.

In some embodiments, a cannula set loading device can include a magazine configured to guide distribution of individual cannula sets. For example, FIG. 12 shows an example embodiment of a magazine 1200 that stores cannula sets 2 in an elongated (straightened) configuration. Each cannula set 2 includes a first needle cap 80 that covers the patient needle and a second needle cap 83 that covers the tube needle. The magazine 1200 can include one or more guiding features, such as a first track 1210 and a second track 1220 configured to engage with and guide first needle caps 80 and second needle caps 83, respectively, of cannula sets 2 that are movable within the magazine. In the orientation shown in FIG. 12, a bottom cannula set 2 can be released from the magazine and dropped via gravity out of the magazine into a loading position. Additionally or alternatively, the bottom cannula set 2 can be released and urged out of the magazine with the aid of a passive force (e.g., from a biasing element such as a spring) and/or an active force (e.g., a motorized actuator). While a vertical orientation of the magazine 1200 is shown in FIG. 12, in other embodiments the magazine 1200 can be in any suitable orientation, such as horizontal.

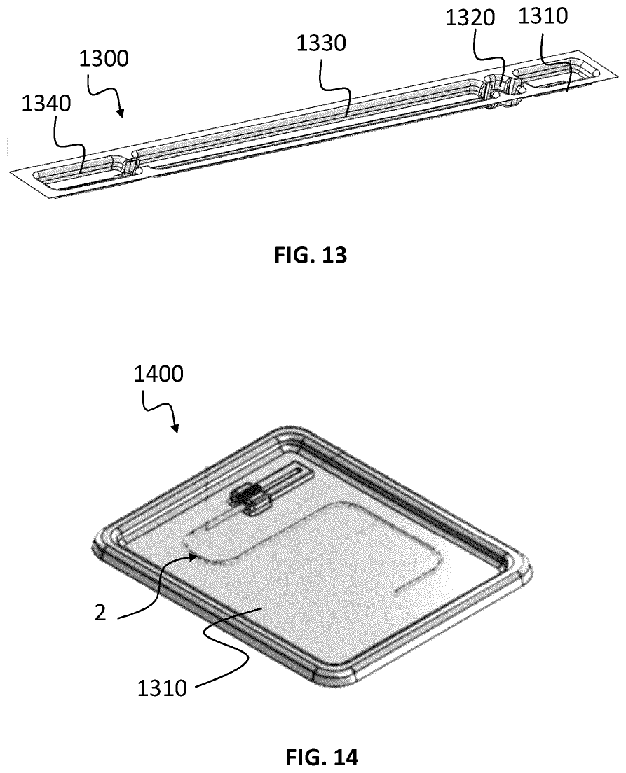

In some embodiments, a cannula set loading device can store cannula sets that are placed and sterilized in individual trays, and the individual trays can be stackable or otherwise collectively stored. For example, FIG. 13 shows an elongated tray 1300 including multiple cavities for receiving various components of a straightened cannula set (not shown), including, for example, a first cavity 1310 shaped and sized to receive a patient needle, a second cavity 1320 shaped and sized to receive a patient needle hub, a third cavity 1330 shaped and sized to receive a tube connecting the patient needle and the tube needle, and/or a fourth cavity 1340 shaped and sized to receive a tube needle hub. As another example, FIG. 14 shows a tray 1400 including a larger tray cavity 1310 for receiving the cannula set 2. In some embodiments, the larger tray cavity 1310 can include a formed recess shaped and sized to house the cannula set 2 in a predetermined configuration (e.g., with a predetermined tube routing, etc.). The trays 1300 and 1400 can, for example, include injection molded or thermoformed material such as plastic or another suitable material. The trays 1300 and 1400 may be sealed with a film and sterilized (e.g., with a sterilant gas such as ethylene oxide) to sterilize the cannula set in preparation for use. In some embodiments, the cannula set can omit first and/or second needle caps, since the sealed trays can function to maintain sterility of the patient needle and/or tube needle during transport and storage.

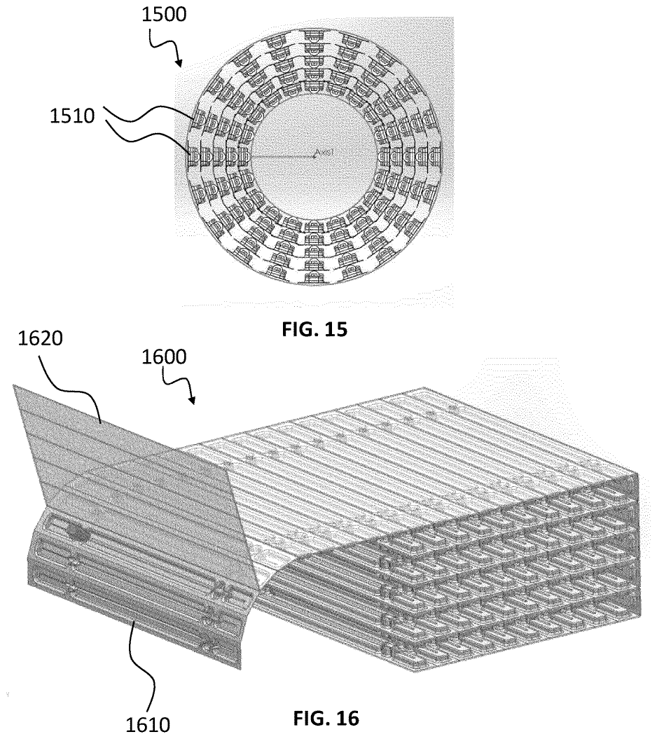

In some embodiments, a cannula set loading device can store cannula sets that are placed and sterilized in individual trays that are joined together in a sheet. For example, FIG. 15 illustrates an example embodiment in which multiple trays 1510, each holding an individual cannula set, are connected and stored as a roll 1500. As another example, FIG. 16 illustrates an example embodiment in which multiple trays 1610, each holding an individual cannula set, are connected in a sheet 1600 that is foldable or stackable. Each of the trays 1510 Or 1610 can, for example, be similar to either of the trays 1300 or 1400 described above with respect to FIGS. 13 and 14, respectively. For example, as shown in FIG. 16, some or all of the trays 1610 can be sealed by a sheet 1620 and sterilized. In some embodiments, individual trays 1510 or 1610 can be detached from the roll 1500 or sheet 1600, respectively, and moved to a loading position for manipulation. Additionally or alternatively, trays can be individually unsealed (e.g., by peeling back the sheet 1620 a predetermined amount) to expose and allow access to the cannula set contents of each tray, one tray at a time.

### Conclusion

Although many of the embodiments are primarily described above with respect to systems, devices, and methods for automatic venipuncture for blood collection, the technology is applicable to other applications and/or other approaches, such as infusion via cannula. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-16.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A cannula set loading unit to load a cannula set into a cannula insertion system, comprising:
a cannula set having at least one patient needle for introduction into a patient,
a cartridge having a predefined location for holding the patient needle in a fixed position with respect to the cartridge.

2. The cannula set loading unit of claim 1, wherein the patient needle comprises a patient needle hub to connect the patient needle to a cannula insertion device of the cannula insertion system.

3. The cannula set loading unit of claim 1 or 2, wherein the cartridge is stackable with a second cartridge.

4. The cannula set loading unit of claim 3, wherein the cartridge comprises one or more snap connection elements to connect the cartridge to the second cartridge.

5. The cannula set loading unit of any one of the preceding claims, further comprising a first needle cap configured to receive at least a portion of the patient needle.

6. The cannula set loading unit of claim 5, wherein the cannula set comprises the first needle cap.

7. The cannula set loading unit of claim 6, wherein the first needle cap is releasably coupled to the cartridge.

8. The cannula set loading unit of claim 5, wherein the cartridge comprises the first needle cap.

9. The cannula set loading unit of any one of claims 5-8, further comprising a tamper indicator between the patient needle and the first needle cap.

10. The cannula set loading unit of claim 9, wherein the tamper indicator comprises a breakable element.

11. The cannula set loading unit of any of the preceding claims, wherein the cannula set comprises a collection tube needle arranged to connect the cannula set to a blood collection tube.

12. The cannula set loading unit of claim 11, wherein a distal end of the collection tube needle and a distal end of the patient needle are oriented in opposite directions.

13. The cannula set loading unit of claim 11 or 12, further comprising a second needle cap configured to receive at least a portion of the collection tube needle.

14. The cannula set loading unit of claim 13, wherein the cannula set comprises the second needle cap.

15. The cannula set loading unit of claim 14, wherein the second needle cap is releasably coupled to the cartridge.

16. The cannula set loading unit of claim 13, wherein the cartridge comprises the second needle cap.

17. The cannula set loading unit of any one of claims 11-16, further comprising a tamper indicator between the collection tube needle and the second needle cap.

18. The cannula set loading unit of claim 17, wherein the tamper indicator comprises a breakable element.

19. The cannula set loading unit of any one of claims 11-18, wherein the cartridge is configured to hold the collection tube needle in a predefined, fixed position with respect to the cartridge.

20. The cannula set loading unit of any of the preceding claims, wherein the cannula set comprises a tube, wherein the patient needle is coupled to a first end of the tube.

21. The cannula set loading unit of claim 20, wherein the cannula set further comprises a connection element coupled to a second end of the tube opposite the first end of the tube.

22. The cannula set loading unit of claim 21, wherein the connection element comprises the collection tube needle.

23. The cannula set loading unit of claim 21 or 22, wherein the connection element comprises a connection element hub to manipulate the connection element.

24. The cannula set loading unit of any one of claims 20-23, wherein the cartridge is configured to hold the tube in a predefined, fixed position with respect to the cartridge.

25. The cannula set loading unit of claim 24, wherein the cartridge is configured to hold the tube in a predefined loop.

26. A cannula set loading device to load the cannula set from the cannula set loading unit of any one of the preceding claims into a cannula insertion system.

27. The cannula set loading device of claim 26, wherein the cannula set loading device comprises a storage for supporting a stack of cannula set loading units.

28. The cannula set loading device of claim 26 or 27, wherein the cannula set loading device comprises a transfer device to transfer a cannula set loading unit from a storage position to a loading position.

29. The cannula set loading device of claim 28, wherein the transfer device comprises a transfer support to support the cannula set loading unit in a predefined position with respect to the transfer support.

30. The cannula set loading device of claim 29, wherein the transfer support comprises one or more alignment elements to align the cannula set loading unit with respect to the transfer support during loading of the cannula set loading unit on the transfer support.

31. The cannula set loading device of claim 29 or 30, wherein the transfer support comprises one or more locking protrusions to releasably lock the cannula set loading unit on the transfer support.

32. The cannula set loading device of any one of claims 26-31, wherein the patient needle comprises a patient needle hub and wherein the cannula set loading device comprises a patient needle hub manipulator arranged to cooperate with the patient needle hub.

33. The cannula set loading device of claim 32, wherein the cannula set comprises a first needle cap, the first needle cap being configured to receive at least a portion of the patient needle, wherein the patient needle hub manipulator is configured to pull the patient needle at least partially out of the first needle cap.

34. The cannula set loading device of any one of the claims 26-33, wherein the cannula set comprises a connection element, wherein the patient needle is connected at a first end of the tube and the connection element is connected at a second end of the tube opposite the first end to connect the tube to a further device, wherein the connection element comprises a connection element hub, wherein the cannula set loading device comprises a connection element hub manipulator arranged to cooperate with the connection element hub.

35. The cannula set loading device of claim 34, wherein the connection element is a collection tube needle arranged to connect the cannula set to a blood collection tube, wherein the connection element hub manipulator is arranged to push the collection tube needle into a needle holder of a blood collection system of the cannula insertion system.

36. A cannula insertion system to insert a cannula into a human or animal body, comprising: a cannula insertion device configured to insert the cannula into the human
or animal body,
at least one cannula set loading unit as claimed in any one of claims 1-25, and
a cannula set loading device to load a cannula set from the at least one cannula set loading unit into the cannula insertion system.
